(19) United States
(12) Patent Application Publication
(10) Pub. No.: US 2010/0069896 A1
(43) Pub. Date: Mar. 18, 2010

(54) APPARATUS FOR PHOTOTHERAPY OF THE SKIN

(76) Inventor: Dietmar Fischer, Erlangen (DE)

Correspondence Address: HAYNES AND BOONE, LLP;IP Section, 2323 Victory Avenue, Suite 700, Dallas, TX, 75219, US

(21) Appl. No.: 12/439,393

(22) PCT Filed: Sep. 1, 2006
(86) PCT No.: PCT/EP06/08570

§ 371 Date: Oct. 1, 2009

(51) Int. Cl. A61B 18/20; A61B 18/18

(52) U.S. Cl. 606/9

(57) ABSTRACT

An apparatus for phototherapy of the skin exhibits a light-source (101) and a light-intensity-modulating element (103) for generating a light-intensity profile (104) with which the skin (105) to be treated is irradiated, whereby abrupt changes in the radiation intensity, in particular at the edges of the irradiated spots, are avoided.

The invention relates to an apparatus for phototherapy of the skin, and in particular to an apparatus for removing skin pigmentations.

Electromagnetic radiation, designated hereinafter as ‘light’, this term also being intended to include radiation with wavelengths that lie outside the region that is visible to the human eye, has found a large number of therapeutic and/or cosmetic applications for treating human skin. In the course of such treatments, large areas are ordinarily irradiated simultaneously, i.e. areas in the region of square millimetres or even square centimetres.

The state of the art is also familiar with apparatuses and processes with which only small skin areas with dimensions from 10 μm to 1000 μm are irradiated in each instance, cf. WO 2004/037068 A2 and WO 2004/037069 A2. Therein, in each instance, spots of light are applied onto the skin with a plurality of light-sources, and holes are selectively ‘burnt’ into the skin with high radiation intensity. The spacing of the holes amounts to between 30 μm and 2000 μm, and there is also provision to reduce the spot density of the spots situated at the edge of the irradiated area, in order to reduce the action of the radiation in the region of transition to the non-irradiated skin area.

However, in relation to the individual radiation spots themselves this state of the art is not familiar with any special control of the distribution of the light intensity in the spot and at its edges. Rather, the radiation intensity has no specially controlled progression along a diagonal section through a radiation spot, and consequently the radiation has the full intensity in the spot and no intensity outside the spot. Especially at the edges of the treated points (spots), with this state of the art it is very difficult to achieve smoothly continuous transitions of the radiation intensity. This state of the art also has the disadvantage that, when generating a plurality of artificial small ‘wounds’ in the skin, either an individual spot is placed temporally in succession at different points on the skin with an elaborate control system or very elaborate optical devices have to be provided, involving a plurality of light-sources and optical focusing elements, in order simultaneously to ‘burn’ a plurality of ‘micro-wounds’ into the skin.

The object underlying the invention is to provide an apparatus for phototherapy of the skin, with which improved medical and/or cosmetic results are achieved. In particular, with the invention a scar-free and colour-independent removal of tattoos is to be possible, and also a so-called skin rejuvenation.

According to the invention, this object is achieved with an apparatus in which there is arranged in the beam path of the light emitted from the light-source at least one intensity-modulating element which generates on and/or in the skin a light-intensity profile with intensity maxima and intensity minima, the intensity of the light changing continuously at least in the region of the intensity minima. Alternatively, in accordance with the invention the intensity may change in steps at least in the region of the edge of the radiation, in particular in a plurality of steps with, in each instance, relatively small step height in comparison with the maximum intensity.

According to the invention, in the edge regions of an intensity maximum, where the intensity approaches a minimum—for example the value ‘0’, a fluid transition of the radiation intensity, i.e. a smooth or stepped tapering-off of each spot at its edges, accordingly occurs. Corresponding remarks apply in the case of radiation lines at the edges thereof.

The light-intensity-modulating element may exhibit varying embodiments, which are described in the dependent claims. Accordingly, the element may have been configured, for example, in the manner of a ‘neutral filter’ (neutral density filter) with varying absorption of light. For example, in the manner of a glass plate, the transmission of which varies, by reason of varying concentration of light-absorbing substances, in location-dependent manner, for example corresponding to a sine function or to another periodic function, so that the transmitted radiation is intensity-modulated in a manner corresponding to this function.

Alternatively, the partially transmitting plate placed in the beam path may also have been modulated in its thickness in such a way that, by reason of the varying distances travelled in the plate, the radiation is absorbed by the plate to a varying degree, depending upon the location of the transit (Beer's law).

The described intensity modulation of the transmitted radiation which finally arrives at the skin to be treated can also be obtained with polarizing filters, the relative angular position of which in the intensity-modulating element has been set variably. With this configuration of the invention, the relative angular position of the polarizing filters can be changed in simple manner by means of electrical signals, so that the generated light-intensity profile is also adjustable in simple manner.

The spacing between the maxima of the light-intensity distribution is preferably within the range from 5 μm to 1000 μm.

The invention also involves an apparatus for removing natural and artificial skin pigmentations with devices for generating light with wavelengths between 1500 nm and 20,000 nm in one or more spots of light with diameters between 10 μm and 1000 μm, devices for directing such spots of light onto the skin, in order to generate a plurality of micro-wounds therein in such a manner that the distance between the spots of light generating the wounds amounts to between 5 μm and 1000 μm, and with devices for generating a continuous, gradual change in the intensity of light at the edge of the spots of light.

Accordingly, the invention also involves a process for removing skin pigmentations with light.

The apparatuses and processes described above according to the invention can be realised in each instance with a single radiation wavelength or, in each instance, with varying radiation wavelengths. For example, with wavelengths between 300 nm (for the treatment of psoriasis and vitiligo, for example) and 20,000 nm (CO₂ laser). By way of light-source, all types of lasers, for example, enter into consideration, including fibre lasers, LEDs, as well as pulsed and other lamps. The apparatuses and processes can be realised with pulsed radiation and with continuous radiation.

In accordance with the invention, with a single intensity-modulating element, for example of the type described above, and with a single light-source (or alternatively also several light-sources) it is possible in simple manner to generate an intensity profile of the radiation to be applied to the skin, which corresponds equally to a ‘radiation-intensity mountain range’ with maxima and minima.

The radiation emitted from the light-source or light-sources can be transmitted to the light-intensity-modulating element with different transmission systems, for example with optical fibres or with other optical means.

With the invention it is possible to introduce a plurality of light spots onto or into the skin without a plurality of light-sources being required for this purpose, and an elaborate arrangement of focusing elements and associated optical devices (cf. the state of the art initially cited) can also be dispensed with.

The intensity-modulating element according to the invention virtually enables a simulation of a plurality of light-sources, such a plurality being required in the absence of said element.

The invention also enables an adaptation of the intensity profile to the desired medical or cosmetic application within wide ranges.

Exemplary embodiments of the invention will be elucidated in more detail in the following on the basis of the drawing. Shown are:

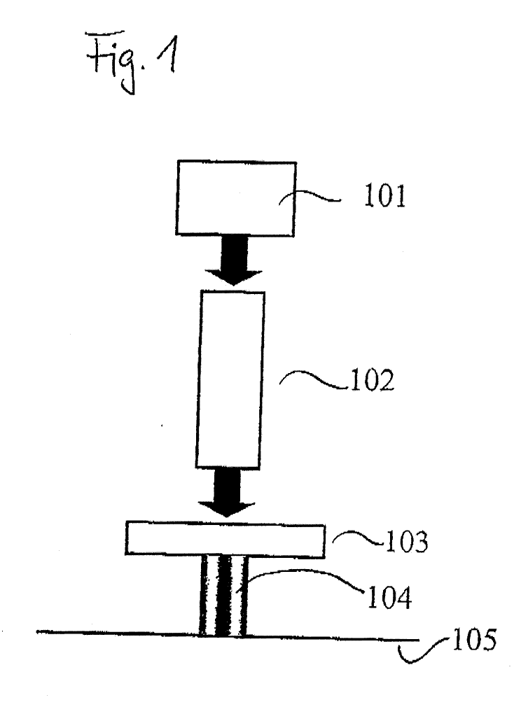

FIG. 1 schematically, a first exemplary embodiment of an apparatus for the ph

[symbol]

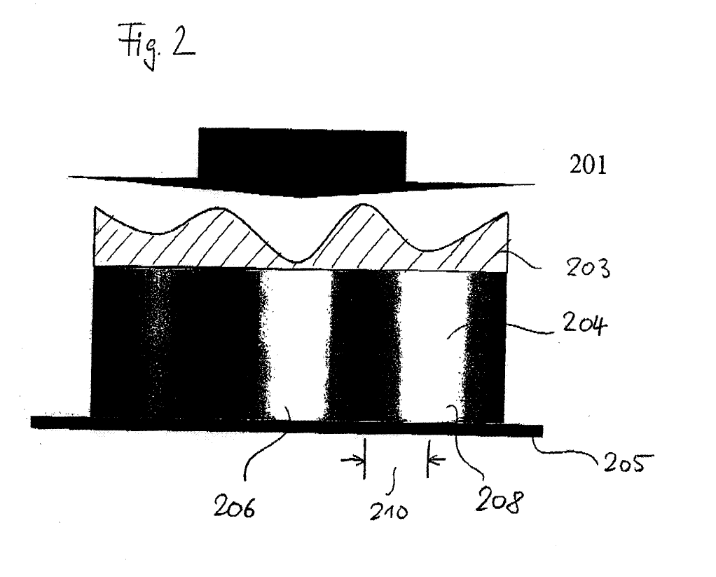

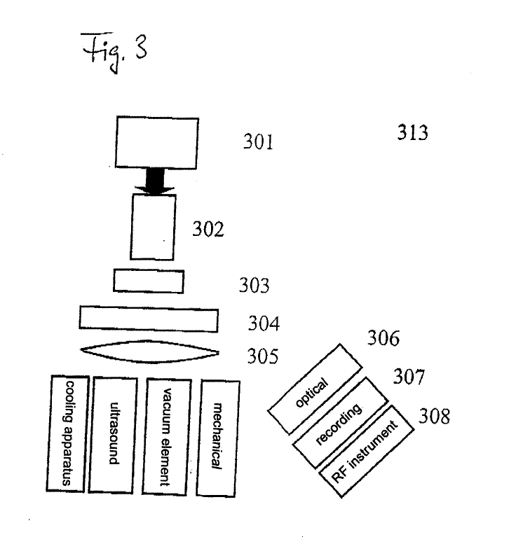

FIG. 2 an exemplary embodiment of a light-intensity-modulating element and the action thereof on the radiation;

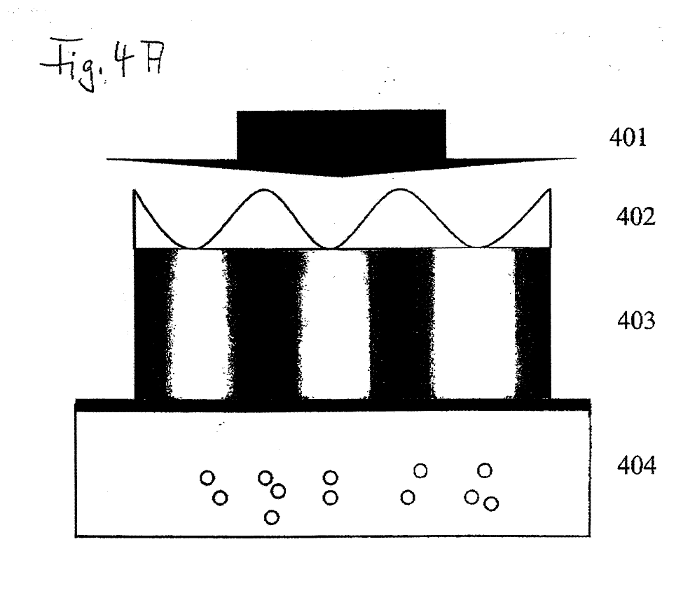

FIG. 3 another exemplary embodiment of an apparatus for the phototherapy of skin, with additional functions;

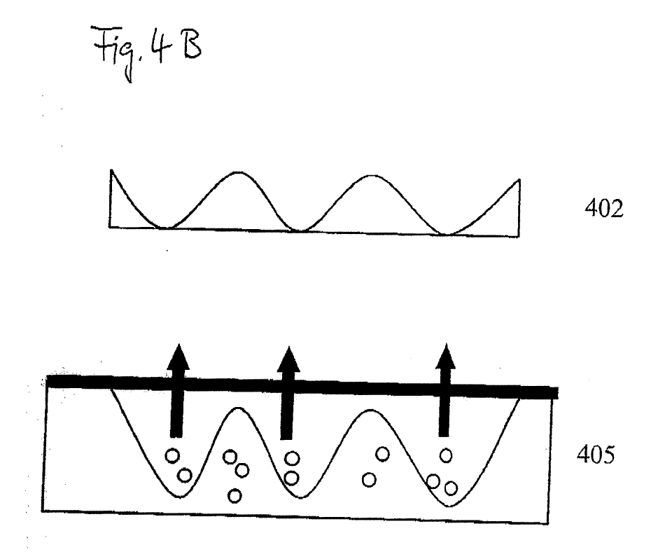

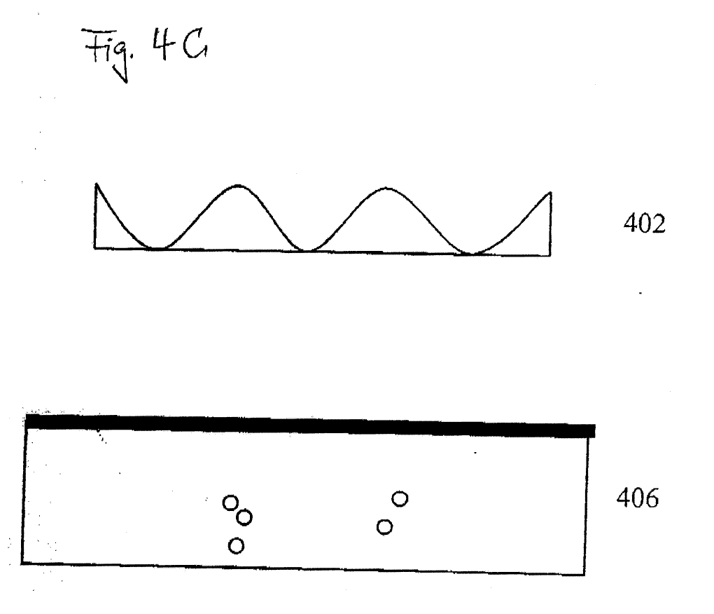

FIG. 4A schematically, the removal of tattoos;

FIG. 4B schematically, the effect of an irradiation for the purpose of tattoo removal in accordance with FIG. 4A; and

FIG. 4C the result of a tattoo removal in accordance with FIGS. 4A and 4B.

The phototherapy apparatus according to FIG. 1 includes a light-source 101, for which the aforementioned radiation-generating apparatuses enter into consideration. Via a transmission device 102, the light emitted from the light-source 101 is transmitted to a light-intensity-modulating element 103 which is represented schematically in FIG. 1. By means of this light-intensity-modulating element 103, the electromagnetic radiation irradiated on this element over a large area—for example, in the region of square millimetres or square centimetres—is spatially modulated in its intensity, i.e. a light-intensity profile arises, with maxima and minima in the manner of a three-dimensional ‘light-intensity mountain range’.

This radiation, with a spatial, continuously or discretely changing light-intensity profile, is introduced onto or into the skin, for example to a depth of approximately 4 mm. The spacing of adjacent minima and maxima of the light-intensity profile lies within the range between 5 μm and 1000 μm.

FIG. 2 shows an exemplary embodiment of a light-intensity-modulating element 203 which, for example, can be employed for element 103 in FIG. 1.

In the case of the exemplary embodiment according to FIG. 2, the radiation 201 emitted from the light-source (not shown in FIG. 2) impinges on the light-intensity-modulating element 203 with a large diameter over the whole surface. The element 203 exhibits, in the manner of a neutral filter (neutral density filter), particles that absorb the light. These particles are homogeneously distributed within the element 203. Consequently, upon the passage of light through the element 203 radiation is absorbed in a manner corresponding to the thickness of the element at the location of the passage of light. The thicker the element 203 at the point in question, the more light is absorbed there. The absorption occurs, in a first approximation, in accordance with an exponential function corresponding to the thickness of the element 203 at the point of the passage of light. Consequently, radiation 204 leaves the light-intensity-modulating element 203 with a light-intensity profile that exhibits maxima and minima. The maxima 206, 208 of the light intensity are represented brightly (white) in FIG. 2. Correspondingly, regions of low intensity in the intensity-modulated light 204 are represented more darkly (grey to black). In places where the light-intensity-modulating element 203 is particularly thin, the light intensity maxima 206, 208 arise. In places where the element 203 is relatively thick, more or less pronounced intensity-profile minima arise as a function of the thickness of the element. Everywhere, and in particular in an edge region—that is to say, where an intensity-profile maximum 206, 208 makes the transition to an intensity-profile minimum, the change in intensity is not abrupt but continuous and gradual. Such a transition region is identified in FIG. 2 by 210. In this region the light-intensity profile changes in accordance with a continuous function, with a gradient which, in particular, does not exceed 80 or preferably 75. According to the invention, a light-intensity profile is generated with gradual, continuous transitions which are smoother than a light-intensity profile that is obtained with a focusing lens, i.e. the intensity gradient, particularly in the region of transition to the intensity minimum, is less steep than in the case of a focusing lens. In this connection, the radiation intensity outside the treatment zone exhibiting the maximum value of the intensity does not necessarily have to diminish to ‘zero’; rather, in these transition regions the intensity may also exhibit a relatively low value of less than 10% of the maximum intensity, or even less than 5% or 3% of the maximum intensity.

FIG. 2 shows a section through the structural elements involved—that is to say, in particular, the light-intensity-modulating element 203. The detail is schematic; substantially more maxima and minima may be provided than are represented. In sections other than that represented—that is to say, for example, sections perpendicular to or angled relative to the plane of the paper—images corresponding to FIG. 2 result, i.e. the minima and maxima of the light-intensity distribution may in a top view (i.e. in a view in the direction of the radiation 201) be substantially round, i.e. maxima and minima arise in the form of ‘spots of light’ or regions in which substantially no light reaches the skin. The spacings between the intensity maxima 206, 208 may be substantially larger than the dimensions of a spot of light. The spots of light themselves may, for example, exhibit diameters between 10 μm and 1000 μm.

With its thickness the light-intensity-modulating element 203 according to FIG. 2 defines the generated intensity distribution on the skin, i.e. the respective local thickness of the element 203 is in each instance inversely proportional to the intensity, corresponding to this location, of the radiation on the skin.

The intensity distribution described above on the basis of FIG. 2 may, in accordance with another exemplary embodiment, be modified to the effect that, instead of a—considered mathematically—continuous transition a stepped change in the intensity is provided in the edge region and in the region of transition to the minimum value of the intensity. A plurality of steps in the transition region are preferably provided. Both exemplary embodiments (continuous and stepped) have in common the fact that the change in the intensity in the edge regions, in which the irradiation makes a transition to a minimum value, does not make a transition from 100% to the minimum value (for example, 0%) abruptly but gradually or in graduated manner in the manner described.

For the light-intensity-modulating element 203 varying configurations are possible in modification of the exemplary embodiment described on the basis of FIG. 2. For instance, several polarizing filters may be arranged in succession in the radiation direction, and their relative angular position may be varied in such a way that the light-intensity profile described above is generated. For this purpose, polarizing filters that are adjustable by means of electrical voltages are also available, which then enable a simple adaptation of the light-intensity profile to different medical or cosmetic applications.

It is also possible to realise the light-intensity-modulating element 203 by extremely small crystals being arranged in a support, said crystals reflecting radiation differently by virtue of a different spatial orientation.

Semi-transmitting mirrors with varying transmissions may also be provided. Various optically excitable media likewise enter into consideration, for example dyestuffs, fluorescent substances, gases, crystals, optical fibres, which, for example by virtue of an inhomogeneous concentration or compositions, generate the light-intensity profile in the manner described above, with spacings between maxima and minima of the order of magnitude from 5 μm to 1000 μm.

Finally, it is also possible to realise an exclusively discrete light-intensity profile with fluid transitions to the non-irradiated regions with an optical grating.

FIG. 3 shows an apparatus for the phototherapy of skin with further functional components, which are only represented schematically in the Figure and which are each available as such to a person skilled in the art but which enable special advantages in interaction with the apparatus according to the invention. The apparatus according to FIG. 3 has, once again, a light-source 301 and a transmission system 302. Upstream of the entry of the light into the light-intensity-modulating element 304 there is arranged a scanner 303 with which the light can be guided over a treatment zone.

A focusing element is 305 is situated in the beam path downstream of the element 304.

With respect to the skin to be treated, a cooling apparatus and/or an ultrasonic instrument and/or a vacuum device and/or mechanical devices for skin deformation (for example, rollers) may be provided. Such assemblies are represented schematically in FIG. 3 by means of blocks. In addition, the apparatus may exhibit optical elements 306 for improving the view of the treatment zone. These optical elements may also exhibit means for obtaining an image of the treatment and recording it—for example, video cameras, magnifying glasses, magnifiers, cameras, projectors. In addition, means 307 may also be provided for recording and for analysing reflected radiation. An RF instrument 308 is likewise capable of being employed. The radiation with a defined intensity profile, introduced into the skin as described above, changes the absorption behaviour of the skin in respect of RF radiation, since changes in the temperature of the skin influence the impedance thereof. In this way, defined regions of the skin can be selectively heated, i.e. treated, in accordance with the generated light-intensity profile.

It is also possible to accommodate the described apparatuses, in particular corresponding to FIGS. 1 and 2, in a hand-held instrument that is capable of being handled comfortably by the operating surgeon. In this connection, devices may have been provided for the purpose of detecting the movement of the handpiece and, in particular, the speed of movement of the handpiece over the skin.

The apparatuses described on the basis of FIGS. 1 to 3 can be generally employed for ablative and non-ablative applications. They are suitable for eliminating pathological or undesirable lesions of the skin. In particular, in the cosmetic field it is possible to remove tattoos of any colour as well as benign pigmented changes in the skin in scar-free manner. This removal will be elucidated in the following:

If light intensities of sufficient strength (fluence) are employed with the apparatuses described above, so-called minimally-invasive wounds such as ‘holes’ are burnt into the skin. The bodily functions react to this with an incrustation from the deeper dermal layers. Contained in this crust are, in addition to the natural healthy pigments, also the pigments of pigmented changes in the skin or the artificially introduced pigments of a tattoo. After a short healing-phase, the crust drops off, and hence the pigments contained therein are also removed.

With the apparatuses described above, given adjustment of suitable intensities other applications are also possible, such as the aforementioned skin rejuvenation—that is to say, in particular, the smoothing of folds and regions of skin unevenness, the reduction of the sizes of pores, the tightening of the tissue (lifting effect) and the general unification of the pigmentations of the skin. The effects that are achievable in this connection are all attributed to the same healing process.

Warts, acne and all types of scars (hypertrophic, atrophic, hypotrophic, acne scars) can also be treated.

Further fields of application are photosensitive skin diseases such as, for example, psoriasis, vitiligo, atopic dermatitis, alopecia areata, mycosis fungoides, depigmented scars and striae, lichen ruber planus and also vascular lesions such as, for example, couperose, teleangiectasias, haemangiomas, port-wine stains, rosacea and, furthermore, cellulite. Likewise conceivable is an application for light-assisted removal of hair.

Conceivable as an application would be, furthermore, for the processing of materials, the micro-inscription of products and components for the purpose of unambiguous labelling and for the purpose of protection against illegal counterfeit products, as well as the generation of fine mechanical and electronic components and 1D, 2D, 3D structures within the μn range by removal or heating of material by means of laser.

In ophthalmology, fine hole structures could be generated in the cornea or in the retina. Analogously, tissues in these structures could, of course, also be appropriately heated.

In particularly preferred manner, the apparatuses and processes according to the invention that have been described can be employed for the purpose of removing pigmentations. This will be described in more detail in the following.

The objective is to remove pigmentations largely without undesirable side-effects. Such undesirable side-effects are, in particular, scars, post-inflammatory hyperpigmentations or hypopigmentations. Open wounds with risks of infection are also to be avoided. Long healing-times are also to be avoided.

With the apparatuses and processes described above, this is achieved as follows.

According to FIG. 4A, light 401 is modulated by a light-intensity-modulating element 402 in the manner described above in such a way that light 403 with a light-intensity profile of the described type is generated. This light is radiated into the skin 404. The pigments therein are indicated in FIG. 4A by means of small circles. For example, radiation with wavelengths between 1500 nm and 20,000 nm is generated and is split up into small spots with diameters between 10 μm and 1000 μm. In this connection, the spots may be arranged in overlapping manner or may also be separated from one another, so that between the spots there is non-irradiated tissue or only slightly irradiated tissue.

With this radiation, as a result of ablation of tissue small channels or holes are generated in the epidermis and dermis which, for example, have diameters within the range from 10 μm to 1000 μm. These channels or holes extend as far as the pigments, for example of the tattoo to be removed. The surrounding tissue outside the channels or holes remains intact.

This state is represented schematically in FIG. 4B. Pigments are partly directly vaporised by the laser beam, partly broken up into smaller pigments, and partly transported to the surface of the skin via the crust arising. After a few days the crust falls off, and with it the pigments to be removed also disappear.

FIG. 4C shows the state attained after this, in which distinctly fewer pigments are present in the skin 406. In a further treatment cycle, analogous to that described above, these residual pigments can also be removed.

The process described above with the described apparatus can be employed not only for the purpose of removing tattoos but also for the purpose of removing epidermal and dermal pigmented lesions, such as, for example, naevus of Ota, naevus of Ito and lentigos.

With the techniques that have been described it is particularly advantageous that arbitrary tattooing colours and also colour combinations can be removed, since the radiation no longer has to be absorbed by the pigments.

The introduction of the micro-holes that have been described causes no visible scars but, on the contrary, at worst invisible micro-scars. The healing-times are also relatively short.

The risk of hypopigmentations and hyperpigmentations is slight. By virtue of the minimal area of the ‘wounds’, the natural barrier function of the skin with regard to infections is preserved, so that the risk of infection is relatively slight.

1.-10. (canceled)

11. Apparatus for the phototherapy of skin, with at least one light-source, whereby in the beam path of the light emitted from the light-source there is arranged at least one intensity-modulating element which generates on and/or in the skin a light-intensity profile with intensity maxima and intensity minima, whereby at least in the region of the intensity minima the intensity of the light changes continuously or in steps,
characterised in that the gradient of the intensity profile at the transition from the maximum to the minimum does not exceed 80.

12. Apparatus according to claim 1, characterised in that the spacing of adjacent maxima of the light-intensity profile is between 5 μm and 1000 μm.

13. Apparatus according to claim 1, characterised in that a single light-intensity-modulating element is arranged in the beam path of the light emitted from the light-source.

14. Apparatus according to claim 2, characterised in that a single light-intensity-modulating element is arranged in the beam path of the light emitted from the light-source.

15. Apparatus according to claim 1, characterised in that the light-intensity-modulating element consists at least partly of light-absorbing material.

16. Apparatus according to claim 1, characterised in that the light-intensity-modulating element exhibits two or more polarizing filters, the relative angular position of which varies within such ranges that in a target area the light-intensity profile exhibits maxima and minima with spacings between the maxima within the range from 5 μm to 1000 μm.

17. Apparatus according to claim 1, characterised in that the light-intensity-modulating element exhibits two or more polarizing filters, the relative angular position of which is capable of being changed by means of electrical signals.

18. Apparatus according to claim 1, characterised in that the light-intensity-modulating element exhibits crystals of varying spatial orientation and/or dimension.

19. Apparatus according to claim 1, characterised in that the light-intensity-modulating element exhibits semi-transmitting mirrors of varying transmission.

20. Apparatus according to claim 1, characterised in that the light-intensity-modulating element exhibits varying optically excitable media in varying concentration and/or composition.

21. Apparatus according to claim 1, characterised in that the light-intensity-modulating element is an optical grating.

22. Apparatus for removing skin pigmentations, with an apparatus according to claim 1, wherein said at least one light source is configured to generate light with wavelengths between 1500 nm and 20,000 nm in one or more spots of light with diameters between 10 μm and 1000 μm, and including devices for directing such spots of light onto the skin, in order to generate therein a plurality of micro-wounds in such a manner that the spacing between the spots of light generating the wounds amounts to between 5 μm and 1000 μm.

23. Apparatus for phototherapy of skin, comprising:
at least one light-source generating a beam of light defining a beam path;
at least one intensity-modulating element positioned within said beam path, said intensity-modulating element controlled to generate a light-intensity profile on or in the skin with an intensity maximum and an intensity minimum; and
whereby the intensity of the light transitions fluidly by a gradient transition from the maximum to the minimum that does not exceed 80.

24. Apparatus for phototherapy of human tissue, comprising:
at least one light-source generating a beam of light defining a beam path;
a means for controlling the light intensity within said beam such that said beam defines a spot on or inside the human tissue having a gradual change between a maximum intensity and a minimum intensity, wherein the transition gradient between the maximum intensity and the minimum intensity does not exceed 80.
